(19) United States
(12) Patent Application Publication
(10) Pub. No.: US 2010/0082020 A1
(43) Pub. Date: Apr. 1, 2010

(54) MEDICAL LASER APPARATUS HAVING CAPACITANCE SENSOR AND LASER EMISSION CONTROL DEVICE

(76) Inventors: Sung Huan GONG, Seoul (KR); Tae Ho HA, Goyang-si, Gyeonggi-do (KR); Jae Seon SEO, Goyang-si, Gyeonggi-do (KR); Sung Woon SHIN, Gunpo-si, Gyeonggi-do (KR)

Correspondence Address: IPLA P.A., 3550 WILSHIRE BLVD., 17TH FLOOR, LOS ANGELES, CA, 90010, US

(21) Appl. No.: 12/519,721

(22) PCT Filed: Sep. 19, 2007
(86) PCT No.: PCT/KR07/04540

§ 371 Date: Jun. 17, 2009

(30) Foreign Application Priority Data
Dec. 27, 2006 (KR) 10-2006-0134672

(51) Int. Cl. A61B 18/20

(52) U.S. Cl. 606/12

(57) ABSTRACT

A medical laser apparatus having a capacitance sensor and a laser emission control device is provided, which can improve the safety by sensing whether a laser handpiece is in contact with a skin using the capacitance sensor and emitting a laser beam only when the handpiece is in contact with the skin, and which can improve the accuracy by making it possible to confirm whether the handpiece stands perpendicular to the skin surface. The medical laser apparatus includes a main body 10, a beam transfer device 20, a handpiece 110 connected to the beam transfer device 20, an approach sensing unit 120 for sensing whether a subject of surgical operation is within a predetermined distance on the basis of a measured capacitance and outputting a sensed signal if it is sensed that the subject of surgical operation is within the pre [symbol] determined distance; and an emission control unit 140 for outputting an emission control signal to a laser emitting device provided in the main body 10 on the basis of the sensed signal received from the approach sensing unit 120. According to the medical laser apparatus, the laser beam is emitted only when the front end part of the handpiece is uniformly in close contact with the skin.

### TECHNICAL FIELD

The present invention relates generally to a medical laser apparatus. More particularly, the present invention relates to a medical laser apparatus having a capacitance sensor and a laser emission control device, which can improve the safety by sensing whether a laser handpiece is in contact with a skin using the capacitance sensor and emitting a laser beam only when the handpiece is in contact with the skin, and which can improve the accuracy by making it possible to confirm whether the handpiece stands perpendicular to the skin surface.

### BACKGROUND ART

Generally, medical laser apparatuses are provided with diverse safety devices for protecting surgical operators and patients, and one of such safety devices is a laser emission control device.

Most conventional laser emission control methods use step switches which operators step on to control the laser emission. Recently, a laser emission control method using a pressure sensor has been proposed, which is disclosed in Korean Patent Unexamined Publication No. 2004-27152.

According to this method, the emission of a laser beam is controlled only when a user uses the pressure sensor in a state that the operator steps on the step switch. This method provides more strengthened protection than the method using only the step switch. That is, the user can get out of danger occurring due to the false operation of the laser apparatus.

However, the conventional laser emission techniques for a medical laser apparatus as described above have the following problems.

The operation of the laser apparatus can be controlled only through the user's grasp of the pressure sensor, and this causes the user great inconvenience.

Although it is preferable that the laser is emitted when the handpiece stands perpendicular to the skin surface and is in close contact with the skin, no technology of judging such a state has been proposed, and thus the operator should always judge the state only by his/her own sensibility.

### DISCLOSURE OF INVENTION

### Technical Problem

Therefore, the present invention has been made in view of the above-mentioned problems, and it is an object of the present invention to provide a medical laser apparatus having a capacitance sensor and a laser emission control device, which can improve the safety by sensing whether a laser handpiece is in contact with a skin using the capacitance sensor having a simple structure and emitting a laser beam only when the handpiece is in contact with the skin.

It is another object of the present invention to provide a medical laser apparatus having a capacitance sensor and a laser emission control device, which can improve the accuracy by sensing whether the handpiece stands perpendicular to the skin surface and emitting a laser beam only when the handpiece stands perpendicular to the skin surface.

It is still another object of the present invention to provide a medical laser apparatus which is suitable to perform a laser operation by forming a hollow portion for passing therethrough the laser beam in a metal plate for measuring the capacitance.

### Technical Solution

In order to achieve the above objects, in one aspect of the present invention, there is provided a medical laser apparatus having a capacitance sensor and a laser emission control device, the medical laser apparatus including a main body, a beam transfer device, and a handpiece connected to the beam transfer device, according to the present invention, which comprises an approach sensing unit for sensing whether a subject of surgical operation is within a predetermined distance on the basis of a measured capacitance and outputting a sensed signal if it is sensed that the subject of surgical operation is within the predetermined distance; and an emission control unit for outputting an emission control signal to a laser emitting device provided in the main body on the basis of the sensed signal received from the approach sensing unit.

The approach sensing unit may include a sensor conductive pole, provided at a front end part of the handpiece, for determining a reference to capacitance measurement; and a capacitance sensor, electrically connected to the sensor conductive pole, for measuring a capacitance between the sensor conductive pole and the subject of surgical operation and outputting the sensed signal to the emission control unit if it is sensed that the subject of surgical operation is within the predetermined distance on the basis of the measured capacitance.

The sensor conductive pole may be a conductive plate having the same shape as the front end of the handpiece.

The sensor conductive pole may be a metal plate having a hollow portion, formed therein, for smoothly passing therethrough a laser beam emitted from the main body to the subject of surgical operation.

The medical laser apparatus according to the present invention may further include a plurality of capacitance sensors; a plurality of sensor conductive poles connected to the plurality of capacitance sensors, respectively; and an AND gate, connected to the plurality of capacitance sensors, for outputting a sensor output signal to the emission control unit only when sensed signals are received from all the plurality of capacitance sensors.

The sensor conductive pole may include two half metal plates symmetrically arranged to smoothly pass therethrough a laser beam emitted from the main body to the subject of surgical operation, and the two capacitance sensors are connected to the half metal plates, respectively.

The sensor conductive pole may include four quarter metal plates symmetrically arranged to smoothly pass therethrough a laser beam emitted from the main body to the subject of surgical operation, and the four capacitance sensors are connected to the quarter metal plates, respectively.

A projecting rim is formed at the front end of the handpiece, and the metal plates are fitted into the projecting rim.

### ADVANTAGEOUS EFFECTS

The medical laser apparatus having a capacitance sensor and a laser emission control device as constructed above senses whether a laser handpiece is in contact with a skin using the capacitance sensor and emits a laser beam only when the handpiece is in contact with the skin, so that the safety of the apparatus is improved.

Also, the medical laser apparatus senses whether the handpiece stands perpendicular to the skin surface and emits a laser beam only when the handpiece stands perpendicular to the skin surface, so that the accuracy of the apparatus is improved.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a perspective view illustrating the whole appearance of a medical laser apparatus having a capacitance sensor and a laser emission control unit according to an embodiment of the present invention;

FIG. 2 is a block diagram schematically illustrating the construction of a medical laser apparatus according to an embodiment of the present invention;

FIG. 3 is a perspective view of main parts of a medical laser apparatus according to an embodiment of the present invention;

FIG. 4 is a sectional view of FIG. 3;

FIG. 5 is a perspective cut-out view of FIG. 3;

FIG. 6 is a perspective view of main parts of a medical laser apparatus according to another embodiment of the present invention;

FIG. 7 is a block diagram illustrating the construction of main parts of a medical laser apparatus of FIG. 6;

FIG. 8 is a perspective view of main parts of a medical laser apparatus according to still another embodiment of the present invention; and

### BEST MODE FOR CARRYING OUT THE INVENTION

Hereinafter, preferred embodiments of the present invention will be described with reference to the accompanying drawings. The matters defined in the description, such as the detailed construction and elements, are nothing but specific details provided to assist those of ordinary skill in the art in a comprehensive understanding of the invention, and thus the present invention is not limited thereto.

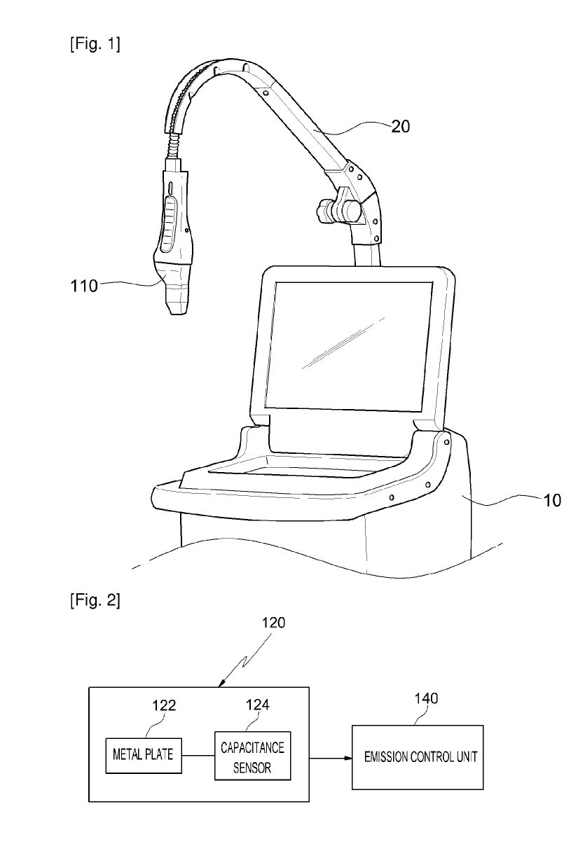

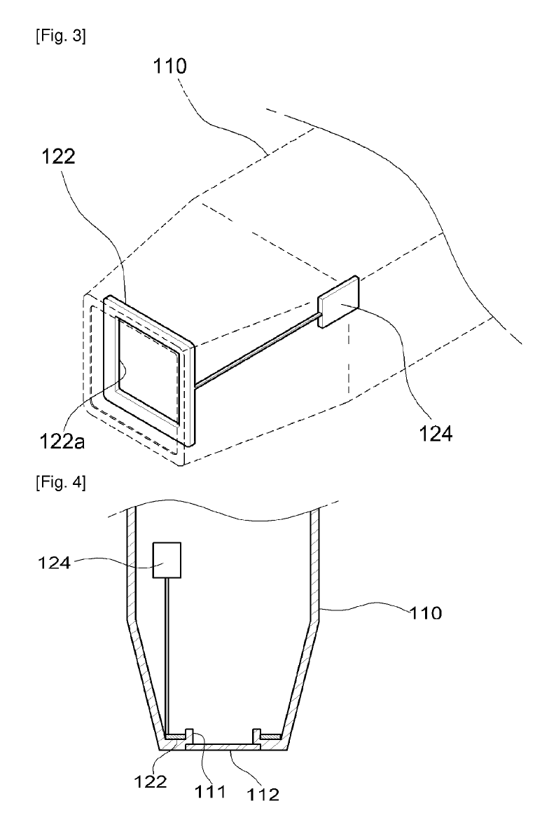

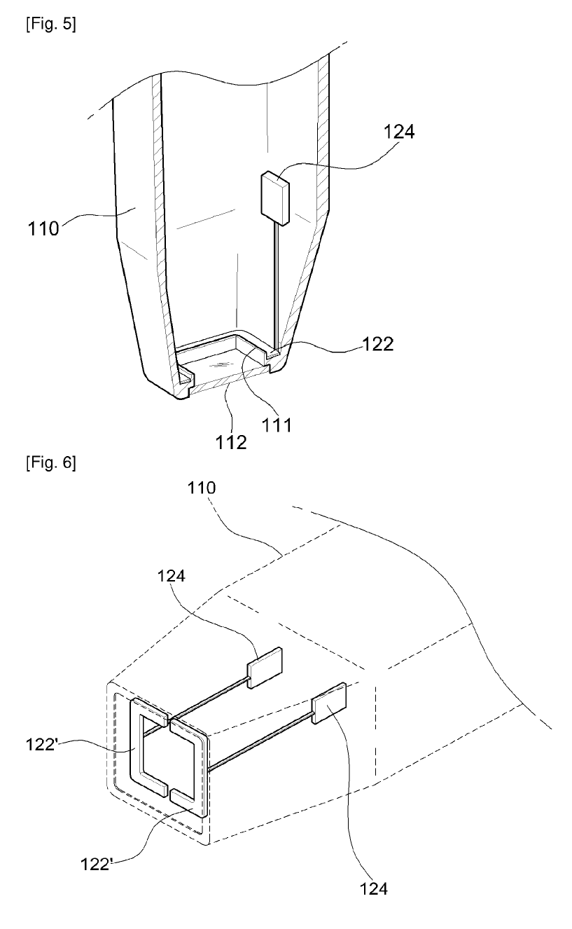

FIG. 1 is a perspective view illustrating the whole appearance of a medical laser apparatus having a capacitance sensor and a laser emission control unit according to an embodiment of the present invention, and FIG. 2 is a block diagram schematically illustrating the construction of a medical laser apparatus according to an embodiment of the present invention. FIG. 3 is a perspective view of main parts of a medical laser apparatus according to an embodiment of the present invention, FIG. 4 is a sectional view of FIG. 3, and FIG. 5 is a perspective cut-out view of FIG. 3.

As illustrated in FIGS. 1 to 5, the medical laser apparatus having a capacitance sensor and a laser emission control unit according to an embodiment of the present invention includes a laser main body 10 for emitting a laser beam, a beam transfer device 20 for guiding the laser beam emitted from the main body 10, a handpiece 110, connected to the beam transfer device 20, for focusing and irradiating the laser beam onto a subject of surgical operation such as a skin, an approach sensing unit 120, and an emission control unit 140.

The approach sensing unit 120 senses whether a subject of surgical operation such as a skin is within a predetermined distance on the basis of a measured capacitance, and outputs a sensed signal to the emission control unit 140 provided in the main body 10 if it is sensed that the subject of surgical operation is within the predetermined distance.

The emission control unit 140 outputs an emission control signal to a laser emitting device (not illustrated) provided in the main body 10 on the basis of the sensed signal received from the approach sensing unit 120.

The approach sensing unit 120 includes a metal plate 122 and a capacitance sensor 124.

The capacitance sensor 124 outputs the sensed signal to the emission control unit 140 provided in the main body 10 if it is sensed that the skin is within the predetermined distance on the basis of the measured capacitance.

The capacitance sensor 124 may be located at any place in the laser system. In the embodiment of the present invention, the capacitance sensor 124 is provided on one inner side of the handpiece 110.

The metal plate 122 is electrically connected to the capacitance sensor 124, and determines a reference to capacitance measurement. The metal plate 122 is provided at a front end part of the handpiece 110 in the same shape as the front end of the handpiece 110, and has a hollow portion 122a, formed therein, for smoothly passing therethrough the laser beam emitted from the main body 10 to the skin.

The hollow portion 122a is formed in order to solve the problem that a space through which the laser beam passes and the position of the metal plate for sensing the capacitance overlap each other.

The metal plate 122 may be in diverse shapes, such as a circle, a tetragon, an ellipse, and so forth, depending on the shape of the front end of the handpiece 110, and the size of the hollow portion 122a may also be varied on the basis of the cross-sectional area of the laser beam.

Now, the structure for mounting the metal plate 122 on the handpiece 100 will be described. At the front end of the handpiece 110, a projecting rim 111 is formed, and the metal plate 122 is fitted into the projecting rim 111.

By fitting the metal plate 122 into the projecting rim 111, the replacement of the metal plate 122 is facilitated.

Further, the metal plate 122 may be adhered to the front end of the handpiece 110 by an adhering means. The adhering means may be implemented by ultraviolet (UV) adhesives.

In FIGS. 4 and 5, the reference numeral 112 denotes a window.

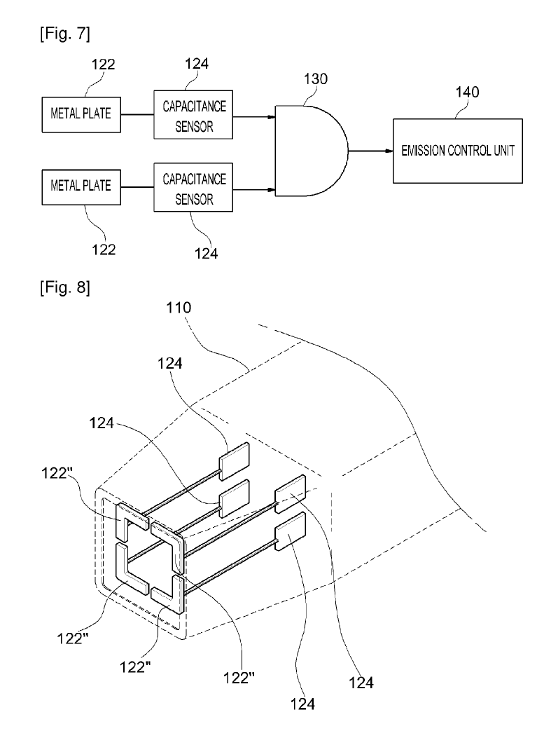

FIG. 6 is a perspective view of main parts of a medical laser apparatus according to another embodiment of the present invention, and FIG. 7 is a block diagram illustrating the construction of main parts of a medical laser apparatus of FIG. 6.

As illustrated in FIGS. 6 and 7, the medical laser apparatus according to another embodiment of the present invention includes two capacitance sensors 124, two half metal plates 122′ connected to the two capacitance sensors 124, respectively, and symmetrically arranged to smoothly pass therethrough the laser beam emitted from the main body 10 to the skin, and an AND gate 130, connected to the two capacitance sensors 124, for outputting a sensor output signal to the emission control unit 140 only when sensed signals are received from both the capacitance sensors 124.

The structure according to another embodiment of the present invention as illustrated in FIGS. 6 and 7 is for the front end of the handpiece 110 to become in close contact with the skin.

In order for the front end of the handpiece 100 to become in close contact with the skin, it is required that the handpiece 110 stands perpendicular to the skin surface. In another embodiment of the present invention, in order to confirm the above-described state that the handpiece 110 stands perpendicular to the skin surface, two half metal plates 122′ are provided as sensor conductive poles, and corresponding capacitance sensors 124 are provided. Only when all the two capacitance sensors 124 connected to the two half metal plates 122′ confirm the contact state, the laser beam is emitted.

FIG. 8 is a perspective view of main parts of a medical laser apparatus according to still another embodiment of the present invention.

As illustrated in FIG. 8, the medical laser apparatus according to still another embodiment of the present invention includes four quarter metal plates 122″ symmetrically arranged to smoothly pass therethrough a laser beam emitted from the main body 10 to the skin, and the four capacitance sensors 124 are connected to the quarter metal plates 122″, respectively.

The structure according to still another embodiment of the present invention as illustrated in FIG. 8 is also for the front end of the handpiece 110 to become in close contact with the skin, and for this, it is required that the handpiece 110 stands perpendicular to the skin surface.

That is, four quarter metal plates 122″ are provided as sensor conductive poles, and corresponding capacitance sensors 124 are provided. Only when all the four capacitance sensors 124 connected to the four quarter metal plates 122″ confirm the contact state, the laser beam is emitted.

The operation of the medical laser apparatus having the above-described construction will now be described.

When the handpiece 110 mounted on the metal plate having a predetermined area approaches an object having a capacitance such as human skin, the metal plate 122 and the human skin form a capacitor having a capacitance.

The capacitor sensor 124 connected by electric wires to the metal plate 122 measures the capacitance between the metal plate 122 and the human skin, and if the measured capacitance exceeds a specified value (i.e., if the metal plate 122 approaches the human skin in a predetermined distance), it is judged that the front end of the handpiece 110 is in contact with the skin, and a specified operation voltage is outputted.

When the emission control unit 140 receives a sensed signal of a specified voltage from the capacitance sensor 124, it controls the laser emitting device (not illustrated) to emit the laser beam.

The laser emission control technique performed by the emission control unit 140 on the basis of the received sensed signal is well known to those skilled in the art, the detailed description thereof will be omitted.

Thereafter, if the handpiece 110 becomes distant from the skin, the laser emission is stopped.

Specifically, when the handpiece 110 becomes far off from the skin, the capacitance of the metal plate 122 for sensing the capacitance is changed. The capacitance sensor 124 senses such a change of capacitance and transmits the sensed signal to the emission control unit 140. The emission control unit 140, having received the sensed signal from the capacitance sensor 124, transmits a control signal for stopping the laser emission to the laser emission device to stop the laser emission.

In the embodiments of the present invention, two or more metal plates are provided to determine whether the handpiece stands perpendicular to the skin surface.

As illustrated in FIGS. 5 and 6, only when the handpiece 110 stands perpendicular to the skin and becomes in close contact with the skin, i.e., only when all the half metal plates 122′ are within the predetermined distance from the skin, the AND gates 130 outputs the sensor output signal to emit the laser beam. Consequently, the laser is emitted only when the handpiece 110 stands perpendicular to the skin surface and is in close contact with the skin without any separation therefrom.

By contrast, if either of the metal plates 122′ is separated from the skin surface, i.e., if the front end of the handpiece 110 does not stand perpendicular to the skin surface and thus is not in close contact with the skin surface, only either of the capacitance sensors 124 outputs the sensed signal. In this case, the AND gate 130 does not outputs the sensor output signal, and thus the laser emission is stopped.

On the other hand, since the operation of the medical laser apparatus according to still another embodiment of the present invention as illustrated in FIG. 8 is similar to that according to another embodiment of the present invention as illustrated in FIGS. 6 and 7, the detailed description thereof will be omitted.

### INDUSTRIAL APPLICABILITY

As can be seen from the foregoing, the medical laser apparatus having capacitance sensor(s) and laser emission control unit according to the embodiments of the present invention has the following effects.

First, since whether a laser handpiece is in contact with a skin is sensed using the capacitance sensor having a simple structure and a laser beam is emitted only when the handpiece is in contact with the skin, the safety of the apparatus is improved.

Second, by forming a hollow portion for passing therethrough the laser beam in metal plates 122, 122′, and 122″ for measuring the capacitance, whether the laser handpiece is in contact with the skin is smoothly sensed.

Third, since whether the handpiece stands perpendicular to the skin surface is sensed and the laser beam is emitted only when the handpiece stands perpendicular to the skin surface, the accuracy of the apparatus is improved.

Fourth, metal plate(s) can be simply and promptly mounted on the handpiece by fitting the metal plates 122, 122′, and 122″ into the projecting rim, and this facilitates the replacement of the metal plates.

While this invention has been described in connection with what is presently considered to be the most practical and preferred embodiment, it is to be understood that the invention is not limited to the disclosed embodiment and the drawings. On the contrary, it is intended to cover various modifications and variations within the spirit and scope of the appended claims.

1. A medical laser apparatus having a capacitance sensor and a laser emission control device, including a main body (10), a beam transfer device (20), and a handpiece (110) connected to the beam transfer device (20), the medical laser apparatus comprising:
an approach sensing unit (120) for sensing whether a subject of surgical operation is within a predetermined distance on the basis of a measured capacitance and outputting a sensed signal if it is sensed that the subject of surgical operation is within the predetermined distance; and
an emission control unit (140) for outputting an emission control signal to a laser emitting device provided in the main body (10) on the basis of the sensed signal received from the approach sensing unit (120).

2. The medical laser apparatus of claim 1, wherein the approach sensing unit comprises:
a sensor conductive pole, provided at a front end part of the handpiece (110), for determining a reference to capacitance measurement; and
a capacitance sensor (124), electrically connected to the sensor conductive pole, for measuring a capacitance between the sensor conductive pole and the subject of surgical operation and outputting the sensed signal to the emission control unit (140) if it is sensed that the subject of surgical operation is within the predetermined distance on the basis of the measured capacitance.

3. The medical laser apparatus of claim 2, wherein the sensor conductive pole is a conductive plate having the same shape as the front end of the handpiece (110).

4. The medical laser apparatus of claim 3, wherein the sensor conductive pole is a metal plate (122) having a hollow portion (122a), formed therein, for smoothly passing therethrough a laser beam emitted from the main body (10) to the subject of surgical operation.

5. The medical laser apparatus of claim 2, further comprising:
a plurality of capacitance sensors (124);
a plurality of sensor conductive poles connected to the plurality of capacitance sensors, respectively; and
an AND gate (130), connected to the plurality of capacitance sensors (124), for outputting a sensor output signal to the emission control unit (140) only when sensed signals are received from all the plurality of capacitance sensors (124).

6. The medical laser apparatus of claim 5, wherein the sensor conductive pole comprises two half metal plates (122′) symmetrically arranged to smoothly pass therethrough a laser beam emitted from the main body (10) to the subject of surgical operation;
wherein the two capacitance sensors (124) are connected to the half metal plates (122′), respectively.

7. The medical laser apparatus of claim 5, wherein the sensor conductive pole comprises four quarter metal plates (122″) symmetrically arranged to smoothly pass therethrough a laser beam emitted from the main body (10) to the subject of surgical operation;
wherein the four capacitance sensors (124) are connected to the quarter metal plates (122″), respectively.

8. The medical laser apparatus of claim 4, wherein a projecting rim (111) is formed at the front end of the handpiece (110), and the metal plates (122, 122′, and 122″) are fitted into the projecting rim (111).

9. The medical laser apparatus of 6, wherein a projecting rim (111) is formed at the front end of the handpiece (110), and the metal plates (122, 122′, and 122″) are fitted into the projecting rim (111).

10. The medical laser apparatus of 7, wherein a projecting rim (111) is formed at the front end of the handpiece (110), and the metal plates (122, 122′, and 122″) are fitted into the projecting rim (111).
